(19) United States
(12) Patent Application Publication
(10) Pub. No.: US 2004/0092855 A1
(43) Pub. Date: May 13, 2004

(54) Self-adhesive wound dressing

(76) Inventor: Tomas Fabo, Molnlycke (SE)

Correspondence Address: YOUNG & THOMPSON, 745 SOUTH 23RD STREET 2ND FLOOR, ARLINGTON, VA, 22202

(21) Appl. No.: 10/381,888

(22) PCT Filed: Sep. 28, 2001
(86) PCT No.: PCT/SE01/02102

(30) Foreign Application Priority Data
Oct. 2, 2000 (SE) 0003536-0

(51) Int. Cl. A61F 13/00; A61F 15/00

(52) U.S. Cl. 602/51

(57) ABSTRACT

The present invention relates to a wound dressing. According to the invention, the dressing comprises a soft, dimensionally stable fat depot (2) which adheres to skin.

### TECHNICAL FIELD

The present invention relates to a wound dressing.

### BACKGROUND TO THE INVENTION

One of the most important functions of the skin is to constitute a two-way barrier which firstly regulates evaporation of water from the body and secondly prevents undesirable substances and particles from penetrating the skin from the outside. For example, the barrier protects against bacteria, fungi, viruses, allergens and toxic substances. The barrier also protects against proteolytic enzymes which can attack the skin when pus or faeces comes into contact with the skin.

The barrier function of skin which surrounds wounds and skin which is affected by various skin diseases is often damaged. The skin barrier is frequently greatly impaired due to the underlying disease or damage. For example, the skin surrounding venous leg ulcers is extremely thin and sensitive. In certain cases, the barrier is impaired still further by the medical treatment. The skin can also be weakened by radiotherapy and cortisone treatment, which often have to be initiated in the case of patients with wounds. In addition, the skin is frequently stressed still further in connection with changes of dressing. In general, the adhesive on many commonly occurring self-adhesive dressings draws with it a layer of corneocytes when the dressings are detached from the skin. When dressings are changed repeatedly, the changes contribute to a weakening of the barrier still further.

Another side effect occurring in connection with wound and skin care is eczema, and as a consequence an impaired barrier function, which is obtained from ointments, plaster adhesive and topical medications. Occlusion is yet another factor which has been shown to weaken the skin barrier. This occurs when a tape or a dressing is adhered to the skin, with the moisture content increasing as the contact surface under the adhesive. After a few days, it can be seen that the skin is moist under the dressing and it is possible to measure an elevated pH, which often has a value of around 7. The lower the vapour permeability of the tape or the dressing, the more rapid and more pronounced does this effect become. After 3-4 weeks of occlusion, an inflammation normally arises due to the increase in moisture content and the increase in pH. This skin, having a high moisture content, has a barrier function which is clearly impaired.

A common treatment of the skin surrounding a wound, when an eczema has occurred or when the barrier function of the skin is impaired in some other way, is to spread fat-containing preparations, for example what is termed a barrier cream, Vaseline, zinc paste or ointments around the wound in order to replace the damaged barrier of the skin. Fungus-inhibiting or bacteria-inhibiting substances are added to certain products. Barrier-stimulating substances of varying activity are added to other products. Cortisone is also a common additive. Zinc, alpha-hydroxyacids, etc., are also common additives.

Wound dressings which require the use of ointments, pastes or creams are more troublesome and more time-consuming since it is necessary to work with more than one product. Another disadvantage of using fat-containing preparations is that they prevent almost all self-adhesive dressings from adhering to the skin. Dressings for wounds where the surrounding skin is being treated with ointments, pastes or creams have to be attached using bandages, or else it is necessary to use dressings which are sufficiently large to enable the plaster to be attached to an area of untreated skin located somewhat outside of the area of treated skin. A further disadvantage of ointments, pastes or creams is that they can be found to be smeary and troublesome to handle. It is difficult to apply the correct quantity and a large excess is often applied when a thin film of the greasy substance would actually be sufficient. While the above-described greasy preparations are usually supplied in tubes, a common application form consists in using what are termed ointment compresses (for example Jelonet from Smith & Nephew). The abovementioned disadvantages apply to both application forms.

One aim of the invention is to remove the disadvantages associated with ointments, pastes, creams and ointment compresses. Another aim is to produce a fat-releasing wound dressing which can contain, or interact with, an absorbent body without, in any noticeable degree, impairing the absorptive ability of the absorbent body. In this present document, wound dressings are also understood as meaning sticking plasters or plasters for treating skin disease.

### SUMMARY OF THE INVENTION

According to the invention, these aims are achieved by means of a wound dressing which is characterized in that it comprises a soft, dimensionally stable fat depot which adheres to skin.

In a preferred embodiment, the fat depot has a softness of 5-20 mm, preferably 7-14 mm and a dimensional stability of more than 80%, preferably 95%. Furthermore, the force of adherence to the skin is between 0.2-3 N, preferably between 0.5-2 N and most preferably between 0.7-1.5 N. The fat depot consists of an adhesive matrix to which one or more fatty substances are added. The adhesive matrix can advantageously consist of a polymer or a mixture of polymers, for example a soft, self-adhering silicone adhesive or a soft hot-melt adhesive. The fatty substance consists of a selection of one or more of paraffin (petrolatum), silicone, lanolin, natural human or animal skin fat components, and natural vegetable fat or oils. A preparation having a pharmaceutical effect and/or a skin-care substance can be added to the adhesive matrix.

### BRIEF DESCRIPTION OF THE FIGURES

The invention will now be described with reference to the attached figures, of which;

FIG. 1 shows a diagram of a cross section of a sticking plaster in accordance with a first embodiment of the invention,

FIG. 2 shows a diagram of an absorbent wound dressing in accordance with a second embodiment of the invention,

FIG. 3 diagrammatically illustrates a method for determining the adherence of a dressing to the skin, and

FIG. 4 shows a measuring cone for use in a penetration test, and

FIG. 5 diagrammatically illustrates a penetration test for measuring softness.

### DESCRIPTION OF EMBODIMENTS

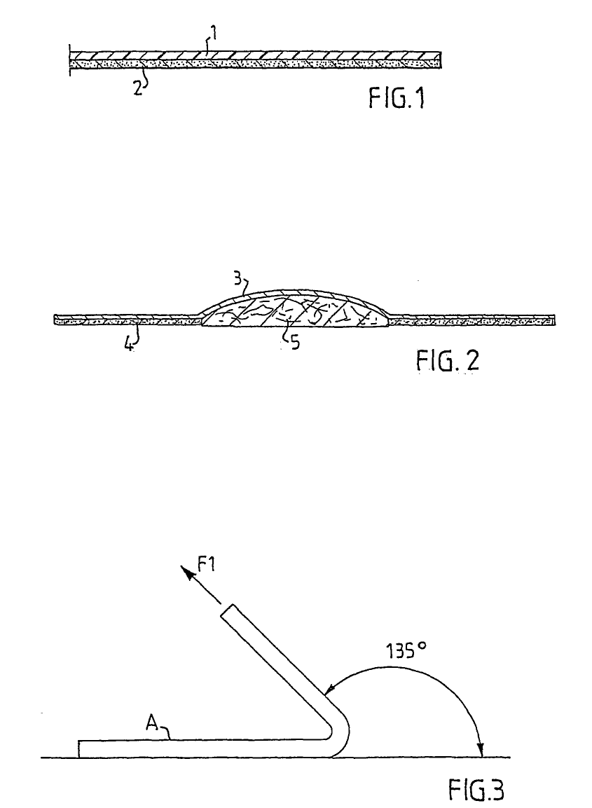

FIG. 1 shows a first embodiment of the invention in the form of a sticking plaster. This plaster consists of a sheet-shaped supporting material 1 one side of which is coated with a soft, self-adhesive fat depot 2.

The supporting material 1 consists of a readily flexed material of the type which is commonly used for sticking plasters, for example a nonwoven fibre fabric, a knitted or woven textile material, a plastic film or the like.

The soft self-adhesive fat depot 2 is made by mixing two components. The larger fraction consists of a form-stabilizing component while the smaller fraction consists of a fatty component. The fatty component can be composed of one or more different types of fatty substances which are used in skin creams, ointments and pastes, such as: paraffin (petrolatum), silicone, lanolin, natural human or animal skin fat components, or natural vegetable fats/oils.

The softness in the fat depot can be achieved either by selecting a form-stabilizing component which is sufficiently soft in itself or by the form-stabilizing component being softened by the addition of a component which is fatty and which thereby forms a soft fat depot.

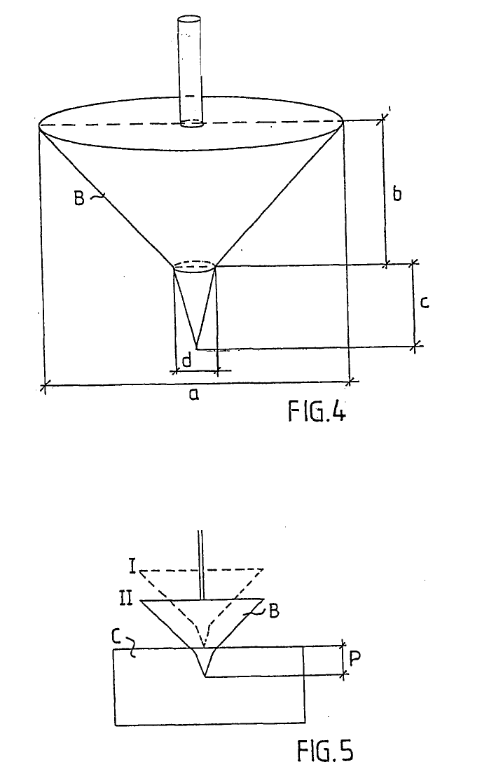

In this present document, “soft” is understood as meaning that a fat depot 2 has a softness of 5-20 mm, preferably 7-14 mm, when measured in accordance with the method illustrated in FIGS. 4 and 5. The softness is measured using a cone B, which weighs 62.5 g and which is allowed to penetrate, by means of gravity, into a 30 mm-thick test piece C of the material of which the fat depot consists. The cone B shown in FIG. 4 has the following dimensions; a=65 mm, b=30 mm, c=15 mm and d=8.5 mm. When the softness is being measured, the cone B is first of all lowered to a position I, which is shown by means of dashed lines in FIG. 5 and in which the tip of the cone precisely touches the surface of the test piece C. After that, the cone B is released so that it can, as a result of gravity, penetrate down into the test piece C. The number of mm by which the tip of the cone has penetrated into the test piece after 5 seconds is measured and constitutes the penetration number P, which is directly proportional to the softness of the test piece. In this present document, the penetration number P is used as a measure of softness.

In this present document, “self-adhesive” is understood as meaning that the fat depot 2 can attach the plaster to normal, non-wet skin by means of adhesion and that the plaster does not fall off due to its own weight. The plaster should be able to be loaded by gravity for at least one hour and at the same time cope with body movements which are not too violent. The force of adherence to skin is measured by the following method, which was developed by the inventor and which is illustrated diagrammatically in FIG. 3. 25 mm-wide strips A of the plaster are applied to the backs of eight individuals and are allowed to remain in place for four hours. After that, they are peeled off at a speed of 25 mm/s and the peeling force F1 is measured. The peeling angle, i.e. the oblique angle which is formed, on peeling, between the surface of the skin and the peeled-off part of the strip A, should be 135°, as shown in FIG. 3. In order to obtain a correctly functioning plaster, the mean value of the force F1 should be 0.2-3 N. The plaster functions very well when the force F1 is between 0.5-2 N, preferably 0.7-1.5 N.

It is also possible to use a 180° peel adhesion test in accordance with ASTM-3330 M-81 for measuring the force of adherence of the dressing or plaster to polished steel plates. The force of adherence to steel which is measured in this way should be between 0.3-4 N, expediently 0.4-3 N, and preferably 0.5-2 N. The major part of the soft, self-adhesive fat depot 2 consists of an adhesive matrix, which is responsible for the dimensional stability. The adhesive matrix can consist of a polymer or a mixture of polymers. In this present document, “dimensionally stable” is understood as meaning that the material has low plasticity, i.e. has a low tendency to flow at body temperature. The very great majority of the deformation of such soft, dimensionally stable adhesives which occurs when the dressings are used normally is of an elastic nature, and the plastic component is relatively insignificant. Neither ointment compresses (for example Jelonet from Smith & Nephew) nor pastes in tubes possess a dimensionally stable fat depot. The dimensional stability can be measured by stretching a test piece lengthwise to 130% of its original length. The test piece is maintained in this stretched position for 1 minute, after which the stretching force is removed. The length of the test piece is then measured after 1 minute. On the one hand, a material whose dimensional stability is sufficiently great copes with this stretching without breaking and, on the other hand, it to a large extent resumes its original length such that, after 1 minute of rest, its length is <110%, preferably <103%, of the original length and, most preferably, <101% of the original length. The size of the test piece is 100 mm×25 mm×5 mm.

Test pieces based on silicone (Elastosil 45554, Wacker-Chemie GmbH, Munich, Germany) containing added ointment and hot-melt adhesive (Dispomelt 70-4647, National Starch & Chemical Company, Bridgewater, N.J., USA) containing added ointment have coped with being stretched to over 200% and then returned to <103% of their original length. These materials have been found to work well as fat depots.

Examples of soft, dimensionally stable, self-adhesive materials are the three two-component addition-curing RTV silicones Q7-2218, 7-9672 and 7-9800 from Dow Corning, Midland, Mich., USA. Other two-component addition-curing RTV silicones are Rhodosil RTV 1507, Rhodia Silicon GmbH, Lübeck, Germany, and Wacker Silicone Elastosil 45554, Wacker-Chemie GmbH, Munich, Germany. Soft, dimensionally stable, self-adhesive materials can also be of the hot-melt adhesive type, for example Dispomelt 70-4647, National Starch & Chemical Company, Bridgewater, N.J., USA, or Dow Corning Bio-PSA Hot Melt Adhesive, Dow Corning, Midland, Mich., USA. Self-adhering adhesives of the type which are used for ordinary self-adhesive dressings, for example acrylate adhesive or EVA-based hot-melt adhesive, can also be suitable adhesive matrices if types are selected which are sufficiently soft or which are softened sufficiently by adding fat.

Experiments were carried out to investigate the adhesion levels of fat depots having different fat contents in order to demonstrate that the adhesion is not lost when fat is added.

Experiment 1. Soft, self-adhesive silicone was prepared by mixing equal parts of the A and B components in the Wacker Silicone Elastosil 45554 (Wacker-Chemie GmbH, Munich, Germany) silicone system. Zinc ointment (Natusan Baby Zinc ointment, Johnson & Johnson AB, 19184 Sollentuna, Sweden) was added and admixed thoroughly using a hand mixer. The mixture was poured out onto a thin, liquid-tight material. The silicone was spread out into a 1 mm-thick layer using a glass rod. A sample was placed in an oven (130° C.) for five minutes and allowed to congeal. After cooling, a polyethene film was placed as a protection on the silicone side. After 24 hours, 25 mm-wide samples were applied to a steel plate and the adhesion force was measured in accordance with ASTM D-3330 M-81.

Results:

| | Adhesion |
|---|---|
| Silicone + 0% zinc ointment | 0.70 N |
| Silicone + 1% zinc ointment | 0.85 N |
| Silicone + 2% zinc ointment | 0.77 N |
| Silicone + 3% zinc ointment | 0.76 N |
| Silicone + 4% zinc ointment | 0.70 N |
| Silicone + 6% zinc ointment | 0.74 N |
| Silicone + 8% zinc ointment | 0.57 N |
| Silicone + 10% zinc ointment | 0.32 N |

The experiment showed that it was possible to admix fat with self-adhesive silicone without the adhesion being lost. The samples were also applied to the skin. None of the samples showed any tendency to come unstuck on its own accord and the samples consequently self-adhered to skin.

Experiment 2. Hot-melt-adhesive samples were prepared by heating the hot-melt adhesive Dispomelt 70-4647 (National Starch & Chemical Company, Bridgewater, N.J., USA) to 150° C. and admixing zinc ointment (Natusan Baby Zinc ointment, Johnson & Johnson AB, 19184 Sollentuna, Sweden) while stirring vigorously. Other samples were prepared by admixing in Vaseline (Chesebrough Klöver Vaseline, Lever Fabergé, Stockholm, Sweden), instead of zinc ointment, while stirring vigorously. The adhesive was spread out on a fibre cloth support in a 1 mm-thick layer. After cooling, a polyethene film was placed as a protection against the adhesive side. After 24 hours, 25 mm-wide samples were applied to a steel plate and the adhesion force was measured in accordance with ASTM D-3330 M-81.

Results:

Adhesion (N)

| | Zinc ointment | Vaseline |
|---|---|---|
| Hot-melt adhesive + 0% zinc | 3.7 | 3.4 |
| ointment or Vaseline | | |
| Hot-melt adhesive + 1% zinc | 2.2 | 3.8 |
| ointment or Vaseline | | |
| Hot-melt adhesive + 2% zinc | 3.7 | 3.6 |
| ointment or Vaseline | | |
| Hot-melt adhesive + 3% zinc | 3.1 | 3.9 |
| ointment or Vaseline | | |
| Hot-melt adhesive + 4% zinc | 2.9 | 2.5 |
| ointment or Vaseline | | |
| Hot-melt adhesive + 6% zinc | 3.7 | 3.0 |
| ointment or Vaselne | | |
| Hot-melt adhesive + 8% zinc | 3.1 | 2.1 |
| ointment or Vaseline | | |
| Hot-melt adhesive + 10% zinc | 2.8 | 2.3 |
| ointment or Vaseline | | |

This experiment, too, showed that it was possible to admix fat with self-adhering hot-melt adhesive without the adhesion being lost. The samples were also applied to skin. None of the samples showed any tendency to come unstuck on its own accord and the samples consequently self-adhered to skin.

While the fatty component is normally present as an emulsion in the dimensionally stabilizing component, it can also be dissolved in the dimensionally stabilizing component. Its function is partly to soften the dimensionally stabilizing component and partly to bring about a fatty layer on the side of the plaster facing the skin. The dimensionally stabilizing component binds the fatty component to a great extent and in the main prevents the fatty component from cold-flowing. This thereby retains the fatty component in the adhesive matrix when the plaster is used such that the fatty layer in contact with the skin is not smeared out to any great extent. The effect of this is that it is possible to produce dressings which are coated with a fat depot without the fat directly escaping out onto other parts of the dressing. In this way, it is possible, for example, to combine fat release with the absorption of pus in a layered dressing which is composed of a fat depot layer and an absorbent layer. The dimensional stability of the depot prevents the fat from spreading out into the absorbent part to such an extent that the absorption is lost. If the fat depot had consisted of a fat depot which was dimensionally unstable, for example paraffin, the paraffin would, to a relatively large extent, have flowed into the absorbent layer in the dressing and decreased its absorptive capacity.

FIG. 2 shows just such a wound dressing which is made up in this way. The wound dressing shown in FIG. 2 comprises a supporting material 3 which, in a central part, supports a wound pad 5 consisting of absorbent material, for example absorbent foam. In addition, a fat depot layer 4 extends on the lower side of the supporting material 3 both in its parts located outside of the wound pad and also on the lower side of the wound pad 5. In this embodiment, the fat depot has to be liquid-permeable, at least in the area beneath the wound pad 5, and can be perforated for this purpose. The fact that the dimensionally stable component binds the fatty substance ensures that the fatty substance does not flow out into the perforations in the fat depot thereby obstructing them. The supporting material 3 and the fat depot 4 correspond to the supporting material 1 and the fat depot 2, respectively, in the embodiment shown in FIG. 1 and are constructed in the same way apart from the perforations which are included in the fat depot.

The function of the fat depot depends on the ratio between the proportions of dimensionally stabilizing component and fatty component. If the proportion of fatty component is too great, the dimensionally stabilizing component cannot bind all the fatty substance and some fat will then leak out of the fat depot. If the proportion of fatty component is too low, the sought-after thin fatty layer will not then form on the surface of the fat depot. The fat depot is preferably formulated with a somewhat larger proportion of fatty component than the dimensionally stabilizing component is able to bind such that the thin fatty layer on the surface of the fat depot can be replaced if this layer is for some reason used up. It has been found that the ratio between the proportion of fatty component and the proportion of dimensionally stabilizing component should be between 0.5:99.5 and 25:75, preferably between 1:99, and 10:90.

The fact that the fat depot is self-adhesive ensures good contact against the skin, and the fatty layer on that side of the fat depot which faces the skin comes to bear closely against the skin.

A prerequisite for the fat depot being self-adhering to skin is that the dimensionally stabilizing component is soft, either because it was originally soft or by becoming softened by means of the fatty component dissolving in the dimensionally stabilizing component. A self-adhesive material which is not sufficiently soft loses its force of adhesion to skin when fat is present on its surface since the softness facilitates the wetting, by the adhesive, of the skin surface, resulting in a large contact area being obtained. The softness also makes the product pliable and comfortable to wear.

Various substances whose purpose is to improve the status of the skin which the fat depot is lying in contact with can be added to the fatty component. The intention is for these added substances to leak out onto the skin from the depot.

The fat depot, which consists of a fatty component and a self-adhesive, dimensionally stabilizing component, consequently possesses three important properties which distinguish it from customary ointment-coated products, namely that of supplying a suitably thick fatty layer to the skin under the fat depot and retaining this fatty layer in the intended location (in other words preventing the fatty component from cold-flowing), that of attaching the dressing, which contains the fat depot, to the skin, and that of ensuring that the fat depot, and thereby the dressing as well, remains at the intended location even when the dressing is subjected to shearing forces and other mechanical stresses which arise during normal use.

In the embodiments shown, the fat depot is only located on the surface of the dressing, in the same way as the adhesive on customary self-adhesive tapes or silicone gel on the surface of Mepilex®, Mölnlycke Health Care AB, Sweden. However, it is also possible to entirely impregnate a product with the fat depot, in the same way as ointment compresses are impregnated with paraffin.

In order to further increase the barrier-strengthening function of a dressing which is provided with a fat depot according to the invention, it is possible to add, to the fat phase, yet more substances which have a positive effect on the health of the skin. Examples of such substances are additives which are present in commercially available ointments, pastes and creams which are used for skin care, or substances which have been reported in the medical literature to have a barrier-strengthening function, for example hydrocortisone, zinc oxide, alpha-hydroxyacids, cholesterol, K+, Ca++, Mg++, pH buffers, fatty acids, urea, vitamins, etc.

The above-described dressings are used for improving the barrier function of skin whose barrier function is damaged or for decreasing the risk of the barrier function being impaired by the dressings which are used. While the invention is first and foremost conceived for being used on skin in connection with wound care, it also functions in all those situations where there is a need to attach a material or a product to the skin.

### IMPLEMENTATION EXAMPLES

### Example 1

Vaseline (Chesebrough Klöver Vaseline, Lever Fabergé, Stockholm, Sweden), zinc ointment (Natusan Baby zinc ointment, Johnson & Johnson AB, 19184 Sollentuna, Sweden), or zinc paste (ACO zinc paste, ACO Hud AB, Stockholm, Sweden), respectively, was added to a self-adhering hot-melt adhesive, Dispomelt 70-4647 (National Starch & Chemical Company, Bridgewater, N.J., USA) at 150° C. and while stirring vigorously. The different mixtures were placed on a Teflon sheet which lay on top of a hot (150° C.) heating plate. The adhesive was spread out in a uniform, approx. 1 mm-thick, layer using a glass rod. The Teflon sheet was moved to a cold underlay, whereupon the adhesive cooled. An SM 810 Sebumeter, Courage and Khazaka Electronic GmbH, Cologne, Germany, was used to measure the sebum value on the upper side of the adhesive both immediately and after 24 hours.

Results:

| | Micrograms/cm², | Micrograms/cm², |
|---|---|---|
| | 0 hr | 24 hr |
| Dispomelt without addition | 47 | 146 |
| Disopmelt + 10% Vaseline | 91 | 210 |
| Disopmelt + 10% zinc ointment | 104 | 203 |
| Disopmelt + 10% Vaseline | 117 | 223 |

It is evident from this experiment that it is possible to add fat to a hot-melt adhesive, resulting in an increase in the release of fat from the surface of the adhesive.

### Example 2

A soft, self-adhesive silicone containing added Vaseline was prepared by admixing Vaseline (Chesebrough Klöver Vaseline, Lever Fabergé, Stockholm, Sweden) into a 1:1 mixture of A and B prepolymers, respectively, of Wacker Silicone Elastosil 45554 (Wacker-Chemie GibH, Munich, Germany). The mixture was stirred thoroughly with a hand mixer and then spread out, in an approx. 1 mm-thick layer, on a Teflon plate. A supporting material consisting of fibre cloth was laid on top, after which the Teflon plate, together with the silicone and the fibre cloth, was placed in a heating oven at 130° C. for 5 minutes. After cooling, the fibre cloth, together with the congealed silicone, was peeled off the Teflon plate. Those samples which were to be tested for the presence of sebum on the surface were dipped into very finely divided quartz powder. The excess of quartz powder was carefully blown off. An SM 810 Sebumeter (Courage and Khazaka Electronic GmbH, Cologne, Germany) was used to measure the Sebumeter value, in micrograms/cm², which is a measure of the quantity of fat on skin, on the surface of the adhesive, i.e. on the lower surface of the samples (=the surface which had been in contact with the Teflon plate) after various times. Samples were also applied to the forearm of an experimental subject for 24 hours, after which the skin Sebumeter value was measured. Results:

| | Sebumeter value on the | | |
|---|---|---|---|
| | adhesive surface, | | |
| | micrograms/cm² | | |
| | 0 hr | 24 hr | 48 hr |
| Silicone without addition | 4 | 47 | 43 |
| Silicone + 1% Vaseline | 6 | 46 | 43 |
| Silicone + 3% Vaseline | 7 | 53 | 53 |
| Silicone + 6% Vaseline | 13 | 42 | 44 |
| Silicone + 10% Vaseline | 13 | 69 | 91 |

| | Sebumeter value on the |
|---|---|
| | skin surface, micrograms/cm² |
| | 24 hr |
| Silicone without addition | 0 |
| Silicone + 1% Vaseline | 1 |
| Silicone + 3% Vaseline | 1 |
| Silicone + 6% Vaseline | 3 |
| Silicone + 10% Vaseline | 25 |

### Example 3

Example 2 was repeated except that zinc ointment (Natusan Baby Zinc ointment, Johnson & Johnson AB, 19184 Sollentuna, Sweden) was added in place of Vaseline. Results:

| | Sebumeter value on the | | |
|---|---|---|---|
| | adhesive surface, | | |
| | micrograms/cm² | | |
| | 0 hr | 24 hr | 48 hr |
| Silicone without addition | 10 | 47 | 46 |
| Silicone + 1% zinc ointment | 11 | 40 | 38 |
| silicone + 6% zinc ointment | 18 | 79 | 85 |
| Silicone + 10% zinc ointment | 34 | 77 | 74 |

| | Sebumeter value on the |
|---|---|
| | skin surface, micrograms/cm² |
| | 24 hr |
| Silicone without addition | 1.6 |
| Silicone + 10% zinc | 5.6 |
| ointment | |

1. Wound dressing, characterized in that it comprises a soft, dimensionally stable fat depot (2; 4) which adheres to skin.

2. Dressing according to claim 1, characterized in that the fat depot (2; 4) has a softness of 5-20 mm, preferably 7-14 mm, and a dimensional stability of less than 110%, preferably less than 103%.

3. Dressing according to claim 1 or 2, characterized in that the force of adhesion to the skin is between 0.2-3 N, preferably between 0.5-2 N, and most preferably between 0.7-1.5 N.

4. Dressing according to claim 1, 2 or 3, characterized in that the fat depot (2; 4) consists of an adhesive matrix to which one or more fatty substances is/are added.

5. Dressing according to claim 4, characterized in that the adhesive matrix consists of a polymer or a mixture of polymers.

6. Dressing according to claim 5, characterized in that the adhesive matrix consists of a soft, self-adhering silicone adhesive or a soft hot-melt adhesive.

7. Dressing according to claim 4, 5 or 6, characterized in that the fatty substance consists of a selection from one or more of paraffin (petrolatum), silicone, lanolin, natural human or animal skin fat components, and natural vegetable fats or oils.

8. Dressing according to claim 4, 5, 6 or 7, characterized in that a preparation having a pharmaceutical effect is added to the adhesive matrix.

9. Dressing according to any one of claims 4-8, characterized in that a skin-care substance is added to the adhesive matrix.
